(19) United States
(12) Patent Application Publication
(10) Pub. No.: US 2024/0008908 A1
(43) Pub. Date: Jan. 11, 2024

(54) Packaging for a Medical Device

(71) Applicant: Clariance, Beaurains (FR)

(72) Inventors: Rodolphe Lourdel, Acq (FR); Pierric Deransart, Sint Martin D'Uriage (FR); Jean Emmanuel Cardon, Domene (FR); Eric Rundstadler, Echirolles (FR)

(73) Assignee: Clariance, Beaurains (FR)

(21) Appl. No.: 18/030,288

(22) PCT Filed: Sep. 27, 2021
(86) PCT No.: PCT/FR2021/051661

§ 371 Date: Apr. 5, 2023

Related U.S. Application Data
(60) Provisional application No. 63/089,826, filed on Oct. 9, 2020.

(30) Foreign Application Priority Data
Feb. 9, 2021 (FR) FR2101226

(51) Int. Cl. A61B 17/86; A61B 17/70; B65D 77/04; B65D 77/26

(52) U.S. Cl. CPC A61B 17/865; A61B 17/7032; B65D 77/0486; B65D 77/26

(57) ABSTRACT

The invention relates to packaging (E, E′) for a medical device (D, 100′), the packaging comprising: —a first container (300, 300′) receiving the medical device (D, 100′), —a second container (400, 400′) receiving the first container (300, 300′) and closed by a closure means (410, 4101), the packaging being characterised in that it comprises a frame (300, 600′) for supporting the medical device (D), the support frame (300, 600′) being preformed so as to have two walls which are arranged opposite each other and between which the medical device (D) is supported by means of at least one transversely arranged movable bearing stop (500, 61 T, 621′).

### FIELD OF THE INVENTION

The invention relates to the field of medical device packaging such as screws or implants and in particular to the adaptations of the packaging making it possible to avoid any contact with the packaged medical device.

### BRIEF DESCRIPTION OF THE PRIOR ART

In the prior art, packagings are known that are specially designed for medical devices such as screws or implants. They are intended to avoid contact between the medical device and the outside as much as possible.

Thus, there is a packaging adopting a trundle configuration and comprising:
a first container closed by a plug and accommodating the medical device in its internal volume;
a second container having an internal volume accommodating the first container, which is closed by a sealed cap serving as a sterile and protective barrier for the first container.

In use, the room nurse opens the second container by removing the cap. The first container then falls onto a sterile field and/or can be handled by a scrub nurse. The user removes the plug and accesses the medical device.

The sterile barrier formed by the second container prevents any contact between the outside and the medical device which remains sterile until the last moment.

The applicant has however found a plurality of disadvantages, including:
the release of the medical device can require complex handling by the scrub nurse and/or require a dedicated tool.
the screw or implant must, in certain situations, be held by the scrub nurse.
the first container is limited in its capacity to accommodate screws of different sizes,
pedicle screws are often intended to be connected to connecting rods. A retaining headless screw is then associated with the head of the preformed pedicle screw to receive and retain the rod. This headless screw ensuring the retention requires a specific packaging or is in some cases accommodated in the plug of the first container, which requires the handling of said plug or of a second packaging.

### BRIEF DESCRIPTION OF THE INVENTION

On the basis of this observation and of pre-established specifications, the applicant conducted research with the aim of handling medical devices without the scrub nurse having to touch said medical device in order to avoid the spread of the bacteria.

This research has resulted in the design and production of a medical device packaging defining a longitudinal axis, said packaging adopting a trundle configuration and comprising:
a first container accommodating the medical device in its internal volume,
a second container having an internal volume accommodating the first container, closed by a closure means and serving as a sterile and protective barrier for the first container.

According to the invention, said packaging is noteworthy in that it comprises an elongate support frame supporting the medical device, said support frame being preformed so as to have two walls disposed opposite each other and between which the medical device is supported by means of at least one movable bearing stop disposed transversely to the longitudinal axis of the medical device.

The movable stop may be a removable stop which separates from the support frame or a stop which moves to enable the screw to be released. This feature is particularly advantageous in that it enables said support to accommodate a plurality of sizes of medical devices. The walls enable the device to be held laterally in the support frame. The stop ensures the axial holding thereof.

Thus, for example in the case of a pedicle screw packaging, the use of a movable stop makes it possible to offer a bearing surface for the screw head having different lengths of threaded part, but having substantially the same head size. This packaging also makes it possible to accommodate screws with different head sizes for the same threaded part.

According to another particularly advantageous feature of the invention, said support frame is preformed from a zone for housing the movable stop.

According to another particularly advantageous feature of the invention, said support frame is preformed with a plurality of zones for housing the movable stop.

The various positioning possibilities of the stop increase the accommodating capacity of the support.

The support frame may be preformed with surfaces serving as a non-movable stop which complements the accommodating possibilities.

Another possibility of accommodating by means of a movable stop according to the invention is implemented by another feature which is noteworthy in that the two walls are preformed from at least one elastic blade with a retaining projection projecting inwards in order to form the movable stop and engaging for retaining purposes with the medical device once the medical device has been disposed between the two walls of the support frame for the purposes of elastic retention. The removability of the stop then consists of a separation of the latter.

When the medical device is a pedicle screw, the two walls are preformed from at least one elastic blade with a retaining projection projecting inwards to form the movable stop and engaging for retaining purposes with the head of the pedicle screw.

This feature engages with an already present feature which is the presence of two lateral orifices initially used for gripping the head of the screw by means of an instrument.

The retaining of the screw by the head makes it possible to use one same frame for a plurality of heads.

According to another particularly advantageous feature of the invention, said support frame forms the first container. Indeed, since the second container forms a sterile and protective barrier, the support can be directly housed in the second container and its walls can serve as a bearing surface for handling purposes without contact with the supported medical device.

The alternative is that according to another particularly advantageous feature of the invention, said support frame is housed in the first container.

Whether it is housed in a first container or whether it itself forms the first container, the support frame of the invention makes it possible to overcome the disadvantages of the devices of the prior art.

It is understood that, according to a preferred application of this packaging, said medical device is a pedicle screw. Thus, according to another particularly advantageous feature of the invention, the head of the pedicle screw bears against the movable bearing stop sliding in at least two parallel grooves preformed in the support frame enabling the different positioning of the bearing stop for the purposes of accommodating screws of different size for the same support frame.

This type of screw is of the type comprising a rod and a head and which is used in combination with a headless screw known as a securing screw.

These headless securing screws are used when the head of the pedicle screw is preformed for the purposes of accommodating an implant rod connecting, for example, two pedicle screws. The headless securing screw holds the connecting rod in position in the head of the pedicle screw. It is therefore advantageous in certain applications to make the securing screw available to the practitioner with the pedicle screw.

Thus, according to a particularly advantageous feature of the invention, said support frame comprises a cavity in which said headless screw is housed. The packaging of the invention thus provides a first container or a support frame containing both the medical device and, in the context of a pedicle screw, also the securing screw with which it engages.

According to another particularly advantageous feature of the invention, said cavity for accommodating the headless screw is equipped with an elastic tongue with a retaining projection which makes it possible, by virtue of its elasticity, to accommodate the headless screw and to hold it in position in the cavity with the projection.

According to another particularly advantageous feature of the invention, said support frame is preformed and sized such that the two walls come together at one end to form a tapered shape which is easily accommodated in the second container.

Naturally, said support frame can be made of a biocompatible material.

Since the fundamental concepts of the invention have been explained above in their most basic form, other details and features will become more clearly apparent upon reading the following description and with reference to the accompanying drawings, which give, by way of non-limiting example, several embodiments of a packaging according to the invention.

### BRIEF DESCRIPTION OF THE DRAWINGS

FIG. 1 is a schematic drawing of a perspective view of an embodiment of a closed packaging according to the invention;

FIG. 2 is a schematic drawing of a perspective view of the partially open packaging of FIG. 1;

FIG. 3 is a schematic drawing of a front view of the packaging of FIG. 1;

FIG. 4 is a schematic drawing of a cross-sectional view along a cross-sectional plane defined in FIG. 3 of the packaging of FIG. 1;

FIG. 5 is a schematic drawing of a perspective view of another embodiment of a closed packaging according to the invention;

FIG. 6 is a schematic drawing of a perspective view of the partially open packaging of FIG. 6;

FIG. 7 is a schematic drawing of a front view of the packaging of FIG. 1;

FIG. 8 is a schematic drawing of a cross-sectional view along a cross-sectional plane defined in FIG. 7 of the packaging of FIG. 5;

FIG. 9 is a schematic drawing of an external view of the first container of the packaging of FIG. 1 accommodating a different screw;

FIG. 10 is a schematic drawing of an external perspective view of the first container of the packaging of FIG. 5.

### DESCRIPTION OF THE PREFERRED EMBODIMENTS

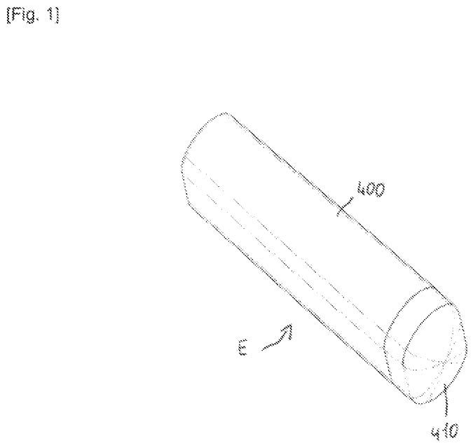

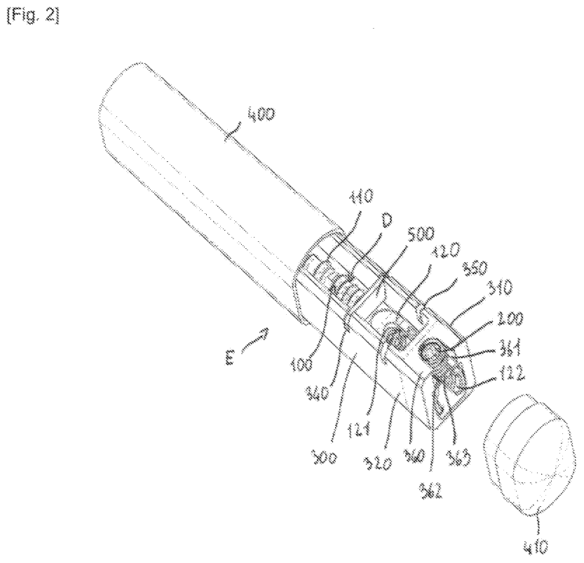

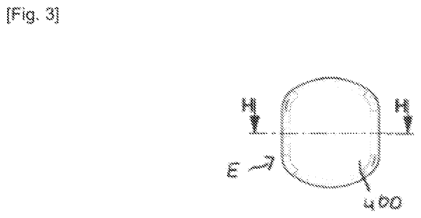

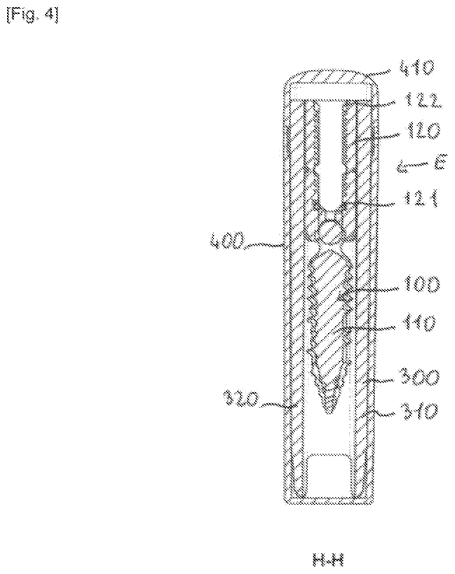

As illustrated in FIGS. 1, 2, 3 and 4, the packaging referenced E as a whole accommodates a medical device referenced D. This medical device, in this case, is formed of a pedicle screw 100 of the type comprising a threaded rod 110 and a head 120 preformed from a groove 121 for accommodating an implant rod (not illustrated) and a tapping 122 for the purposes of accommodating a securing screw 200. The screw 100 defines a longitudinal axis.

The packaging E adopts a trundle configuration and comprises:
a first container 300 accommodating the pedicle screw 100 in its internal volume,
a second container 400 having an internal volume accommodating the first container 300, closed by a closure means, in this case, a plug 410, serving as a sterile and protective barrier for the first container 300.

According to the invention, it is a packaging which avoids any direct contact of the practitioner with the implant, in this case, with the pedicle screw 100. In use, the second container 410 is opened by removing the plug 400. The first container 300 then falls onto a sterile field and/or can be handled by a scrub nurse without touching the screw 100. Tools already known (not illustrated) make it possible to engage with the screw so as to remove it from the first container 300.

In this embodiment, the first container 300 slides in the hollow core of the second container 400.

This first container 300 forms an elongate support frame supporting the pedicle screw 100 having a substantially quadrangular profile. This support frame 300 is preformed so as to have two longitudinal walls 310 and 320 disposed opposite each other and between which the screw 100 is positioned and supported. According to the illustrated embodiment, a third wall 330 connects the two walls 310 and 230 at one of their longitudinal edges. One face of said support frame with a quadrangular profile is perforated and makes it possible to see the screw 100 disposed inside it. The profile formed by the assembly of these walls 310, 320, 330 is open at its two ends. The perforated face accommodates, at the level of the free longitudinal edges of the walls 320 and 310, two transverse grooves 340 and 350. The groove 340 accommodates a movable stop 500, which is removable in this case.

The depth of the grooves 340 and 350 and the dimensions of the stop 500 make it possible to offer a transverse bearing surface for the head 120 of the screw 100. This bearing surface makes it possible to position the screw 100, such that the end of the tapped part 122 of its head 120 opens out at the open end of the support frame 300 so as to be accessible for a tool (not illustrated).

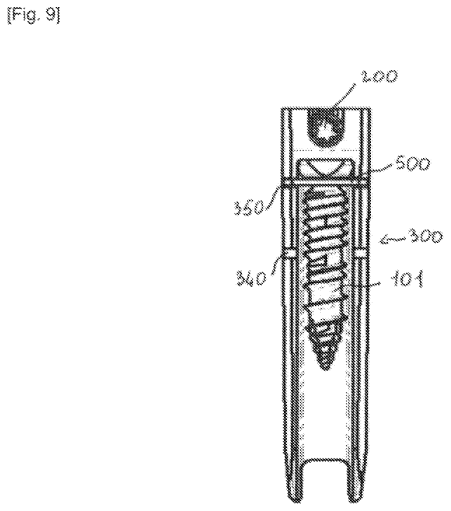

According to the aim of the invention, it is understood that, while ensuring this access to the head, the same support frame 300 can accommodate:
with the stop 500 remaining in the same groove 340, screws with different lengths of threaded parts and with identical heads,
with the stop 500 positioned in the groove 350, screws of different lengths and heads (see FIG. 9 for the screw 101).

As illustrated, the end of the support frame 300 accommodating the head 120 of the screw 100 is preformed so as to prevent the accommodated head from rotating.

Another feature of this support frame 300 relates to the accommodation of the securing screw 200. According to the illustrated embodiment, the support frame is preformed from a wall portion 360 facing the wall 330. The outer surface of this portion 360 is preformed from a cavity 361 provided in its bottom with an elastic tongue 362 equipped at its free end with a retaining projection 363. The cavity 361 is preformed to retain the screw 200 axially. The elasticity of the tongue 362 enables the screw 200 to be accommodated radially and ensures that it is held in position in the cavity by means of the projection 363, which then forms a radial stop.

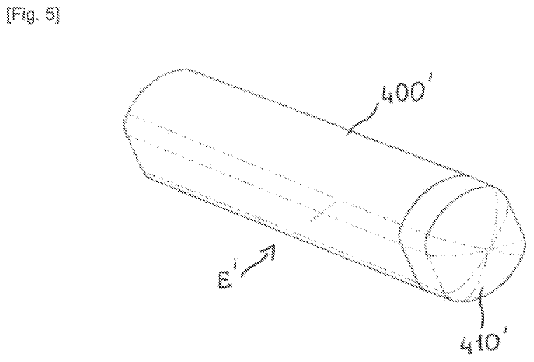

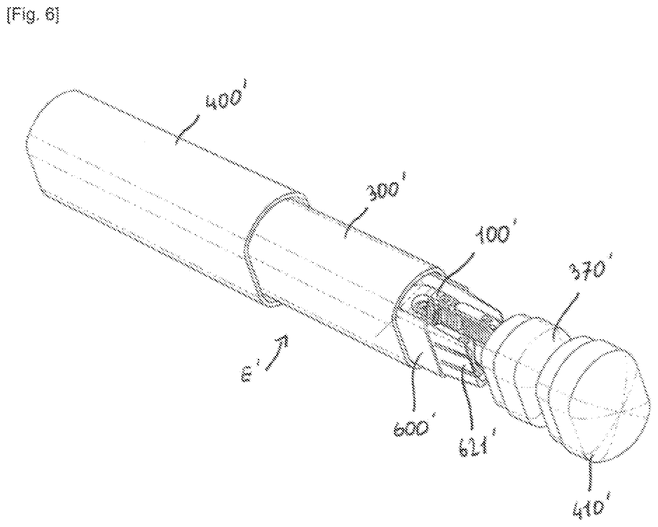

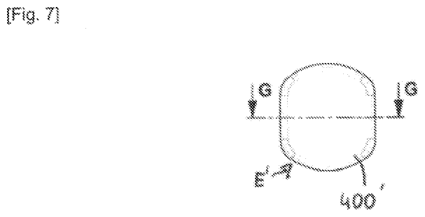

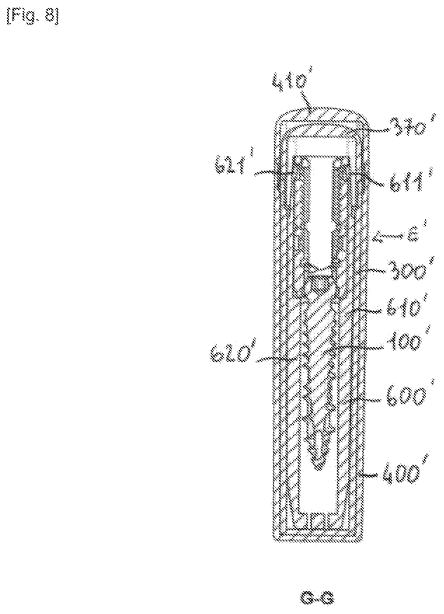

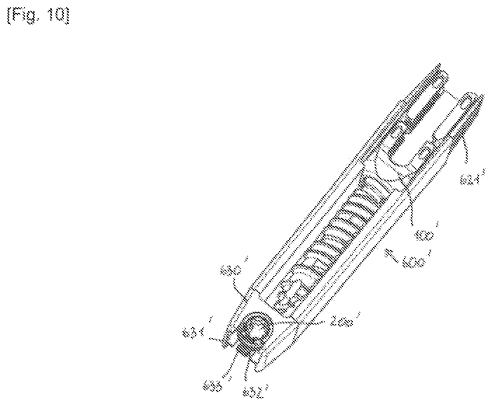

As illustrated in FIGS. 5, 6, 7, 8 and 10, the packaging E′ adopts as the packaging E, a trundle configuration and comprises:
a first container 300′ accommodating the pedicle screw 100′ in its internal volume,
a second container 400′ having an internal volume accommodating the first container 300′, closed by a closure means, in this case, a plug 410′ serving as a sterile and protective barrier for the first container 300′.

The packaging E′ differs from the packaging E in that the support frame 600′ is housed in the first container 300′ the opening of which is controlled by a closure means, in this case, a plug 370′.

This support frame 600′ differs from the support frame 300 of the packaging E in that the two facing walls 610′ and 620′ that form it are each preformed from an elastic blade with a retaining projection 611′ and 621′ projecting inwards to form the movable stop and engaging with the head 120′ of the pedicle screw 100. Once the screw is disposed between the two walls 610′ and 620′ of the support frame 600′, the stops which have moved apart to accommodate it return to position, due to the elasticity of the blades 611′ and 621′ and retain it, creating a transverse bearing surface.

Another feature of this support frame 600′ relates to the accommodation of the securing screw 200′. According to the illustrated embodiment, the support frame 600′ is equipped with a preformed end 630′ of a housing 631′ provided in its bottom with an elastic tongue 632′ equipped at its free end with a retaining projection 633′. The housing is preformed to retain the screw 200′ axially. The elasticity of the tongue 632′ enables the screw 200′ to be accommodated radially and ensures that it is held in position in the housing by means of the projection 633′, which then forms a radial stop.

It is understood that the packagings described and shown above are for the purpose of disclosure rather than of limitation. Naturally, various arrangements, modifications, and improvements can be made to the above examples, without going beyond the scope of the invention.

1. Packaging for a medical device defining a longitudinal axis, said packaging adopting a trundle configuration and comprising:
a first container accommodating the medical device in its internal volume,
a second container having an internal volume accommodating the first container, closed by a closure means and serving as a sterile and protective barrier for the first container, CHARACTERIZED IN THAT IT comprises an elongate support frame supporting the medical device, said support frame being preformed so as to have two walls disposed opposite each other and between which the medical device is supported by means of at least one movable bearing stop disposed transversely to the longitudinal axis of the medical device.

2. Packaging according to claim 1, CHARACTERIZED IN THAT said support frame is the first container.

3. Packaging according to claim 1, CHARACTERIZED IN THAT said support frame is housed in the first container.

4. Packaging according to claim 1, CHARACTERIZED IN THAT said support frame is preformed with a plurality of housing zones for the movable stop.

5. Packaging (E, E′) according to claim 1, CHARACTERIZED IN THAT said medical device is a pedicle screw.

6. Packaging according to claim 5, wherein the pedicle screw is of the type comprising a rod and a head and which is used in combination with a headless screw, CHARACTERIZED IN THAT said support frame comprises a cavity in which said headless screw is housed.

7. Packaging according to claim 6, CHARACTERIZED IN THAT said accommodating cavity of the headless screw is equipped with an elastic tongue with a retaining projection enabling by its elasticity the accommodation of the headless screw and holding it in position in the cavity with the projection.

8. Packaging according to claim 5, CHARACTERIZED IN THAT the head of the pedicle screw bears on the movable bearing stop sliding in at least two parallel grooves preformed in the support frame.

9. Packaging according to claim 1, CHARACTERIZED IN THAT the two walls are preformed from at least one elastic blade with a retaining projection projecting inwards in order to form the movable stop and engaging with the medical device, once the medical device has been disposed between the two walls of the support frame for the purposes of elastic retention.

10. Packaging according to claim 5, CHARACTERIZED IN THAT the two walls are preformed from at least one elastic blade with a retaining projection projecting inwards to form the movable stop and engaging with the head of the pedicle screw.

11. Packaging according to claim 1, CHARACTERIZED IN THAT said support frame is preformed and sized such that the two walls come together at one end to form a tapered shape easily accommodated in the second container.

12. Packaging according to claim 9, CHARACTERIZED IN THAT the two walls are preformed from at least one elastic blade with a retaining projection projecting inwards to form the movable stop and engaging with the head of the pedicle screw.

13. Packaging according to claim 6, CHARACTERIZED IN THAT the head of the pedicle screw bears on the movable bearing stop sliding in at least two parallel grooves preformed in the support frame.

14. Packaging according to claim 7, CHARACTERIZED IN THAT the head of the pedicle screw bears on the movable bearing stop sliding in at least two parallel grooves preformed in the support frame.

15. Packaging according to claim 2, CHARACTERIZED IN THAT said support frame is preformed with a plurality of housing zones for the movable stop.

16. Packaging according to claim 3, CHARACTERIZED IN THAT said support frame is preformed with a plurality of housing zones for the movable stop.

17. Packaging according to claim 2, CHARACTERIZED IN THAT the two walls are preformed from at least one elastic blade with a retaining projection projecting inwards in order to form the movable stop and engaging with the medical device, once the medical device has been disposed between the two walls of the support frame for the purposes of elastic retention.

18. Packaging according to claim 3, CHARACTERIZED IN THAT the two walls are preformed from at least one elastic blade with a retaining projection projecting inwards in order to form the movable stop and engaging with the medical device, once the medical device has been disposed between the two walls of the support frame for the purposes of elastic retention.

19. Packaging according to claim 4, CHARACTERIZED IN THAT the two walls are preformed from at least one elastic blade with a retaining projection projecting inwards in order to form the movable stop and engaging with the medical device, once the medical device has been disposed between the two walls of the support frame for the purposes of elastic retention.

20. Packaging according to claim 5, CHARACTERIZED IN THAT the two walls are preformed from at least one elastic blade with a retaining projection projecting inwards in order to form the movable stop and engaging with the medical device, once the medical device has been disposed between the two walls of the support frame for the purposes of elastic retention.
